(19) United States
(12) Patent Application Publication
(10) Pub. No.: US 2025/0146603 A1
(43) Pub. Date: May 8, 2025

(54) PORT CONNECTORS

(71) Applicant: IDEATE Medical, Inc., Town & Country, MO (US)

(72) Inventors: Tom Hacker, Hood River, OR (US); Philippe Conseil, Montaigu-Vendee (FR); William Wong, Encinitas, CA (US); Florencia Chicatun, Saint-Laurent (CA)

(73) Assignee: IDEATE Medical, Inc., Town & Country, MO (US)

(21) Appl. No.: 18/931,488

(22) Filed: Oct. 30, 2024

Related U.S. Application Data
(60) Provisional application No. 63/595,410, filed on Nov. 2, 2023.

(51) Int. Cl. F16L 21/00; A61L 2/26

(52) U.S. Cl. CPC F16L 21/002; A61L 2/26; A61L 2202/123; A61L 2202/24

(57) ABSTRACT

An insertable port connector for connecting to a fluid port of a device to a sterilization apparatus. The port connector includes a connector body and a porous member. The connector body has an insertion portion, a connection portion, and a porous member brace. The insertion portion is insertable into a lumen of the fluid port and has an inlet. The connection portion connects to the sterilization apparatus. The connection portion has an outlet in fluid communication with the inlet and is configured to be fluidly coupled to the sterilization apparatus. The porous member is supported by the insertion portion. The porous member has a porous structure forming a plurality of minute passageways. The porous member is arranged to be inserted into the lumen with the insertion portion. The porous member is biased outward by the porous member brace so that the porous member engages an interior of the fluid port.

### CROSS-REFERENCE TO RELATED APPLICATION

The present application claims priority to U.S. Provisional Application No. 63/595,410, filed Nov. 2, 2023, the entirety of which is hereby incorporated by reference.

### FIELD

The present disclosure generally relates to port connectors, and, more specifically, to port connectors for sterilizing the surfaces of a fluid port.

### BACKGROUND

Certain articles, such as medical devices (e.g., endoscopes), need to be sterilized between uses. These articles can include interior lumens, which require sterilization. One way to sterilize these interior lumens is to move a sterilization fluid or sterilant through the lumens. In order to move the sterilization fluid through the lumen, a port connector is typically coupled to a fluid port of the article.

For example, PCT Publication No. WO 2018/090133 describes a sterilization system where an endoscope is placed within a chamber with a port connector attached to a fluid port of the endoscope. The port connector fluidly couples the interior lumen(s) of the endoscope with a pressure source (e.g., negative pressure source). To sterilize the interior lumen(s) of the endoscope, a sterilization fluid (such as hydrogen peroxide vapor) is introduced into the chamber and then drawn through the interior lumen(s) of the endoscope via the pressure source fluidly coupled to endoscope by a port connector.

### SUMMARY

In one aspect, a port connector for connecting to a fluid port of a device to be sterilized to a sterilization apparatus. The fluid port has a distal end defining a fluid port outlet. The fluid port defines a lumen extending proximally from the fluid port outlet. The port connector comprises a connector body having an insertion portion, a connection portion, and a porous member brace. The insertion portion is sized and shaped to be inserted into the lumen via the fluid port outlet. The insertion portion has an inlet arranged to be disposed in and in fluid communication with the lumen. The connection portion is configured to connect to the sterilization apparatus. The connection portion has an outlet in fluid communication with the inlet and is configured to be fluidly coupled to the sterilization apparatus. A porous member is supported by the insertion portion of the connector body. The porous member has a porous structure defining a plurality of minute passageways. The porous member is arranged to be inserted into the lumen via the fluid port outlet with the insertion portion. The porous member is biased outward by the porous member brace so that the porous member engages an interior of the fluid port.

In another aspect, a port connector for connecting to a fluid port of a device to be sterilized to a sterilization apparatus. The fluid port has a distal end defining a fluid port outlet. The fluid port defines a lumen extending proximally from the fluid port outlet. The port connector comprises a connector body having an insertion portion and a connection portion. The insertion portion is sized and shaped to be inserted into the lumen via the fluid port outlet. The insertion portion has an inlet arranged to be disposed in and in fluid communication with the lumen. The connection portion is configured to connect to the sterilization apparatus. The connection portion has an outlet in fluid communication with the inlet and is configured to be fluidly coupled to the sterilization apparatus. The connector body has a plurality of alignment projections arranged to be disposed in the lumen and engage an interior of the fluid port to resist withdrawal of the insertion portion from the lumen. A porous member is supported by the insertion portion of the connector body. The porous member has a porous structure defining a plurality of minute passageways. The porous member is arranged to be inserted into the lumen via the fluid port outlet with the insertion portion.

In another aspect, a port connector for connecting to a fluid port of a device to be sterilized to a sterilization apparatus. The fluid port has a distal end defining a fluid port outlet. The fluid port defines a lumen extending proximally from the fluid port outlet. The port connector comprises a connector body having an insertion portion and a connection portion. The insertion portion is sized and shaped to be inserted into the lumen via the fluid port outlet. The insertion portion has an inlet arranged to be disposed in and in fluid communication with the lumen. The connection portion is configured to connect to the sterilization apparatus. The connection portion has an outlet in fluid communication with the inlet and is configured to be fluidly coupled to the sterilization apparatus. The connector body has an insertion stop arranged to engage the fluid port at an interface to stop movement of the port connector relative to the fluid port when the insertion portion is being inserted into the lumen of the fluid port. The insertion stop is shaped to have point contact at the interface with the fluid port. A porous member is supported by the insertion portion of the connector body. The porous member has a porous structure defining a plurality of minute passageways. The porous member is arranged to be inserted into the lumen via the fluid port outlet with the insertion portion.

Other objects and features of the present disclosure will be in part apparent and in part pointed out hereinafter.

### BRIEF DESCRIPTION OF THE DRAWINGS

FIG. 1 is a perspective of a port connector according to one embodiment of the present disclosure connected to a fluid port;

FIG. 2 is a cross-section of the fluid port;

FIG. 3 is a perspective of the port connector;

FIG. 4 is a perspective of a connector body of the port connector;

FIG. 5 is a cross-section of the port connector connected to the fluid port;

FIG. 6 is an enlarged, fragmentary view of FIG. 5;

FIG. 7 is a perspective of a bevel profile;

FIG. 8A a perspective of a port connector according to another embodiment of the present disclosure;

FIG. 8B is a cross-section of the port connector of FIG. 7A connected to a fluid port;

FIG. 9A a perspective of a port connector according to another embodiment of the present disclosure;

FIG. 9B is a cross-section of the port connector of FIG. 8A connected to a fluid port;

FIG. 10 is a perspective of a port connector according to another embodiment of the present disclosure connected to a fluid port;

FIG. 11 is a perspective of the port connector of FIG. 10;

FIG. 12 is a perspective of a connector body of the port connector of FIG. 10;

FIG. 13 is a cross-section of the port connector of FIG. 10 connected to the fluid port;

FIG. 14 is an enlarged, fragmentary cross-section of the connector body of the port connector of FIG. 10; and

FIG. 15 is an enlarged, fragmentary perspective of an alignment projection of the port connector of FIG. 10.

Corresponding reference characters indicate corresponding parts throughout the drawings.

### DETAILED DESCRIPTION

The port connectors disclosed herein can be used with the sterilization system and method described in PCT Publication No. WO 2018/090133. The port connectors described herein enable the fluid ports themselves to be sterilized even with a port connector attached thereto. The port connectors permit the sterilization fluid to come into contact with interior and/or exterior surfaces of the fluid ports that would otherwise be blocked or covered using conventional port connectors, thus enabling generally the entire fluid port to be sterilized, and more specifically the exposed surfaces of the fluid port. The port connectors of the present disclosure are insertable port connectors that are inserted into the fluid ports (e.g., into the end of the lumen defined by the fluid ports) while permitting the sterilization fluid to come into contact with interior surface(s) of the fluid ports that define a portion (e.g., end portion) of the lumen. The port connectors disclosed herein may be referred to as leaky connectors because the port connectors may not form an absolute fluid tight seal with the fluid port.

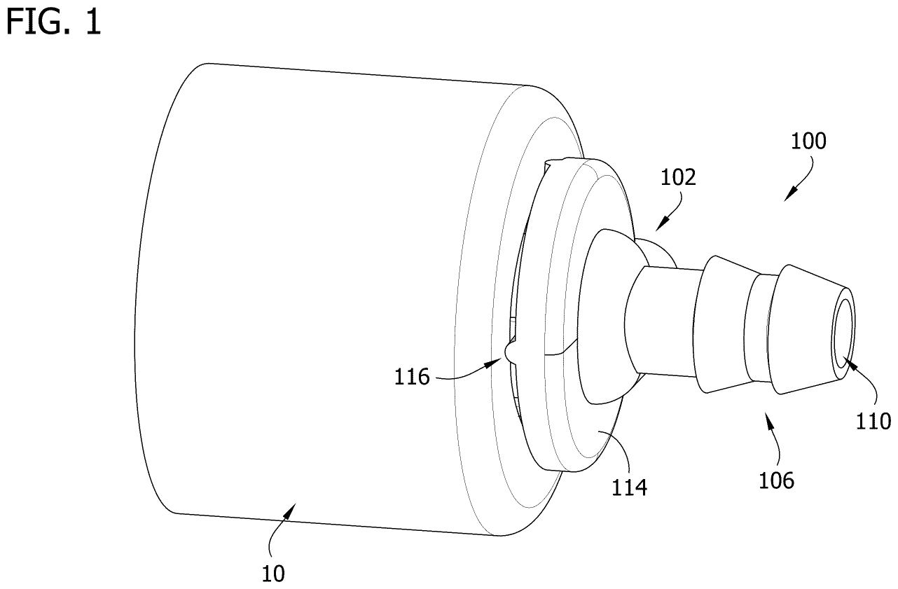

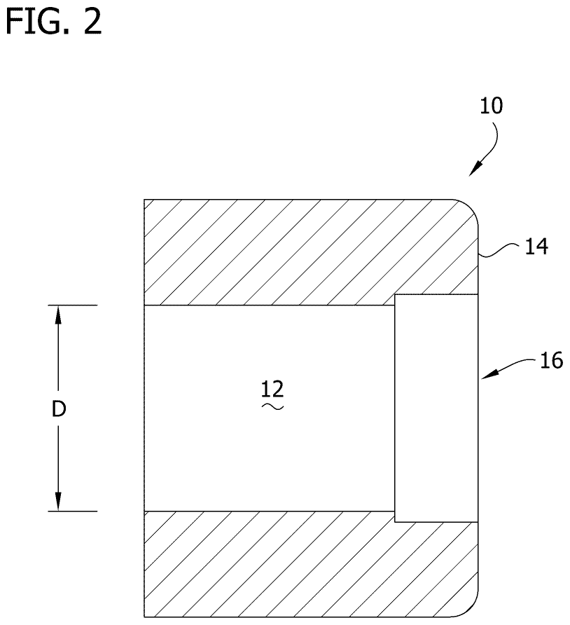

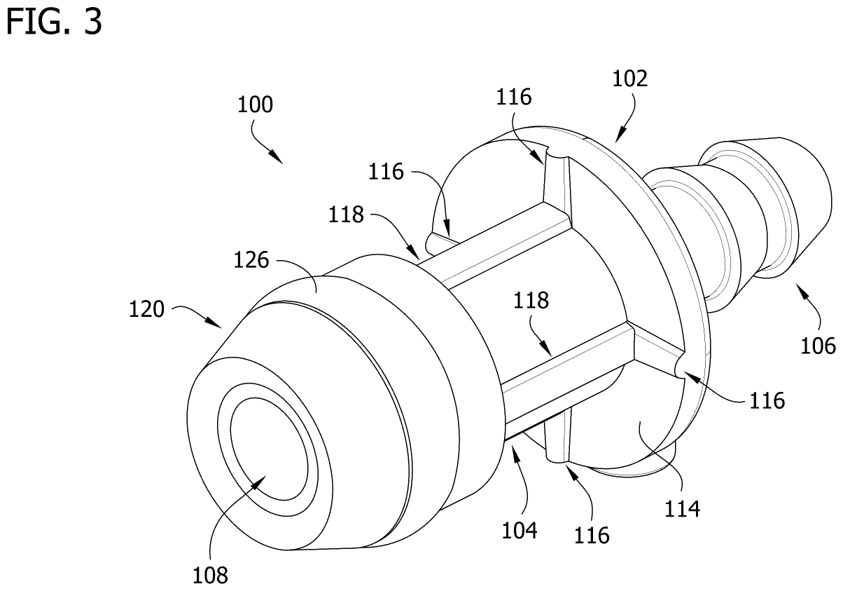

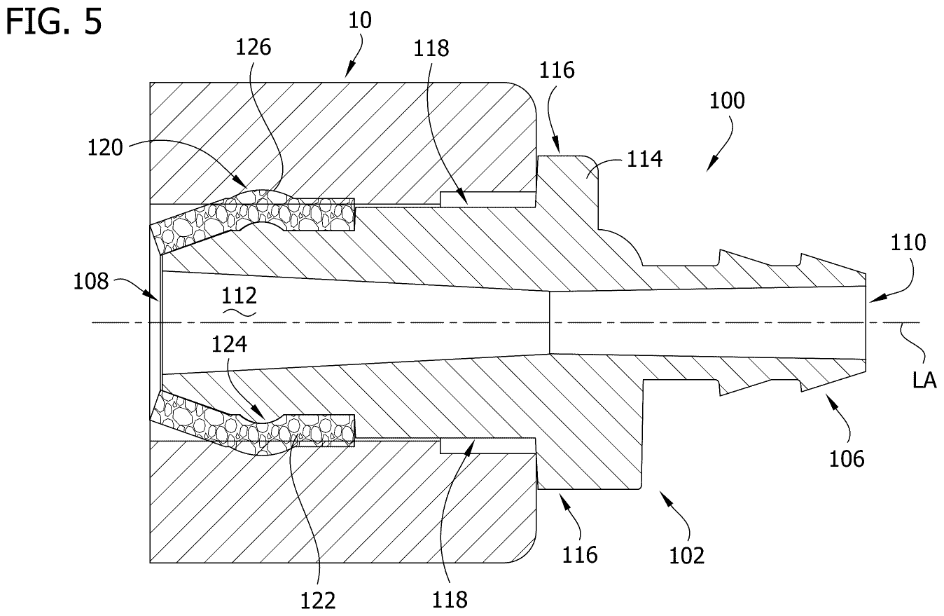

Referring to FIGS. 1-6, one embodiment of a port connector according to the present disclosure is generally indicated by reference numeral 100. The port connector 100 is shown attached to a fluid port 10 of an article (not shown), such as an endoscope. The illustrated fluid port 10 is a female fluid port. As shown in FIG. 2, the fluid port defines a fluid port outlet 16 at the end (e.g., distal or free end) thereof and a lumen 12 extending proximally from the fluid port outlet. The lumen 12 extends along the article and is open at the end opposite the fluid port 10. The lumen 12 may also branch out and have a plurality of opens ends opposite the fluid port 10. The fluid port 10 has a distal end surface 14 (broadly, an exterior surface) at the end. The distal end surface 14 faces distally and borders the fluid port outlet 16. The fluid port 10 includes one or more inner cylindrical surfaces which bound and define the lumen 12. Other configurations of fluid ports may be used without departing from the scope of the present disclosure.

The port connector 100 is used for connecting the fluid port 10 of an article or device to be sterilized (via a sterilization fluid) to a pressure source (e.g., a negative pressure source and/or a positive pressure source) of a sterilization system or apparatus (not shown), such as the sterilization system described in PCT Publication No. WO 2018/090133. In one embodiment, the pressure source is a negative pressure source that creates a pressure differential to draw fluid (e.g., a sterilization fluid) from the environment surrounding the article and the port connector 100, into interior lumen(s) of the article and through the fluid port 10 and the port connector to sterilize the interior surfaces (e.g., interior lumen(s)) of the article. The negative pressure source can be any suitable pressure source such as a vacuum, a pump, or a chamber having a lower pressure then the environment surrounding the article. As used herein, the phrase “negative pressure” means a pressure that is lower than a pressure of the environment surrounding the relative component(s) to which the negative pressure is being applied, such as the port connector 100. For example, applying negative pressure from a negative pressure source to the port connector 100 means the negative pressure source imparts a low pressure on the port connector relative to the pressure in the environment surrounding the port connector (e.g., the chamber of the sterilization system in which the port connector is positioned). In other words, the negative pressure creates a negative pressure differential between the environment surrounding the relative components and the negative pressure source, thereby causing fluid to flow from the environment toward the negative pressure source. A negative pressure can be a pressure at or above atmospheric pressure or a pressure below atmospheric pressure (vacuum). In certain preferred embodiments, a negative pressure is below atmospheric pressure (vacuum).

The port connector 100 includes a connector body 102. The connector body 102 has an insertion portion 104 and a connection portion 106. The insertion portion 104 forms the proximal end of the connector body 102. The insertion portion 104 is sized and shaped to be inserted into the lumen 12 of the fluid port 10, via the fluid port outlet 16. The insertion portion 104 has or defines an inlet 108 (e.g., a connector body inlet). The inlet 108 is arranged such that it is disposed in the lumen 12 of the fluid port 10 and is in fluid communication with the lumen of the fluid port when the port connector 100 is connected to the fluid port (e.g., when the insertion portion 104 is inserted into the lumen). This allows fluid, such as sterilization fluid to flow from the lumen 12 into the inlet 108 of the connector body 102. The inlet 108 is at the proximal end of the insertion portion 104. In the illustrated embodiment, the insertion portion 104 has a generally cylindrical shape, although the insertion portion can have other shapes without departing from the scope of the present disclosure. The insertion portion 104 is tapered at the proximal end, to facilitate insertion of the insertion portion into the fluid port 10.

The connection portion 106 is distal of the insertion portion 104. The connection portion 106 forms the distal end of the connector body 102. The connection portion 106 has or defines an outlet 110 (e.g., a connector body outlet). The outlet 110 is configured to be fluidly coupled to the negative pressure source (broadly, the sterilization apparatus). The connection portion 106 is configured to connect to the negative pressure source (broadly, the sterilization apparatus). In the illustrated embodiment, the connection portion 106 comprises a barbed hose fitting configured to connect to a hose or conduit (e.g., be inserted into an open end of the hose) of the negative pressure source. This fluidly couples the outlet 110 to the negative pressure source applying the pressure differential across the port connector 100. The outlet 110 is fluidly coupled to the inlet 108. The connector body 102 includes a fluid passageway 112 (e.g., lumen, bore) extending between and fluidly coupling the inlet 108 and the outlet 110. The insertion and connection portions 104, 106 each define a portion of the fluid passageway 112. The connector body 102 (broadly, the port connector 100) has a longitudinal axis LA extending between the inlet 108 and the outlet 110. The fluid passageway 112 extends along the longitudinal axis LA.

The connector body 102 includes a flange 114. The flange 114 is disposed at an intermediate position between the proximal and distal ends of the connector body. In the illustrated embodiment, the flange 114 is generally disposed between the insertion and connection portions 104, 106. The flange 114 is orientated generally perpendicular to the longitudinal axis LA. The connector body 102 may include one or more (e.g., a plurality of) insertion stops 116. In the illustrated embodiment, the connector body 102 includes four insertion stops 116 equally spaced part around the longitudinal axis, although more or fewer insertion stops are within the scope of the present disclosure. Each insertion stop 116 is arranged to engage the fluid port 10 to stop further insertion of the insertion portion 104 into the lumen 12 (e.g., stop further proximal movement of the port connector 100 relative to the fluid port 10, or set the position of the insertion portion in the lumen of the fluid port). Thus, the insertion stops 116 set the position of the insertion portion 104 in the lumen 12. Each insertion stop 116 is mounted on and extends generally proximally from a proximal face of the flange 114. The insertions stops 116 are each shaped to have only line contact or point contact with the fluid port 10. The insertion stops 116 are not shaped to have face-to-face contact with the fluid port 10. It has been discovered that the sterilization fluid is able to penetrate the interface created by two elements when the two elements are in line contact (e.g., the elements only contact each other along a line) or when the two elements are in point contact (e.g., the elements only contact each other at a single point). This enables the sterilization fluid to sterilize all the exposed areas of the two elements, including the portions in contact with one another (e.g., the portions creating the line or point contact). In contrast, the sterilization fluid is not able to penetrate (e.g., entirely penetrate) the interface created by two elements in face-to-face contact (e.g., two planar surfaces engaging one another, or two matching curved surfaces engaging one another). Thus, the sterilization fluid cannot completely sterilize the portions of elements in face-to-face contact with one another.

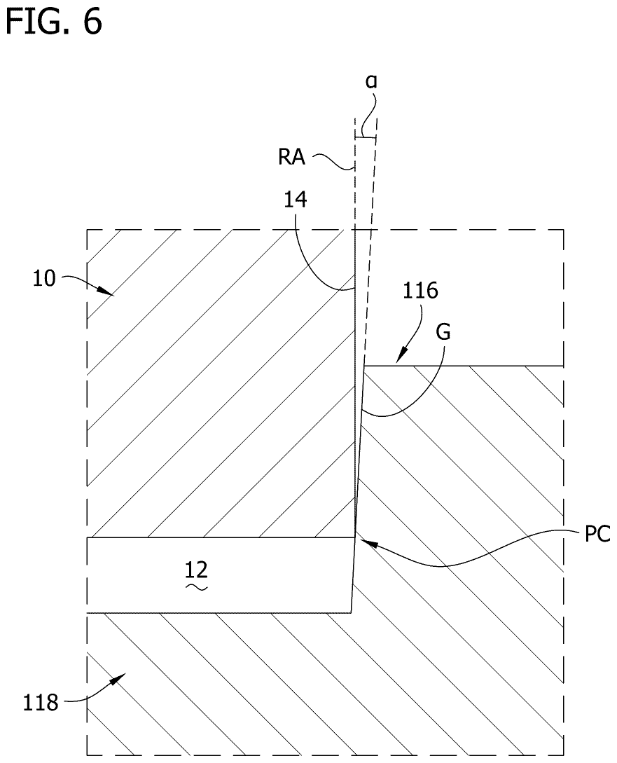

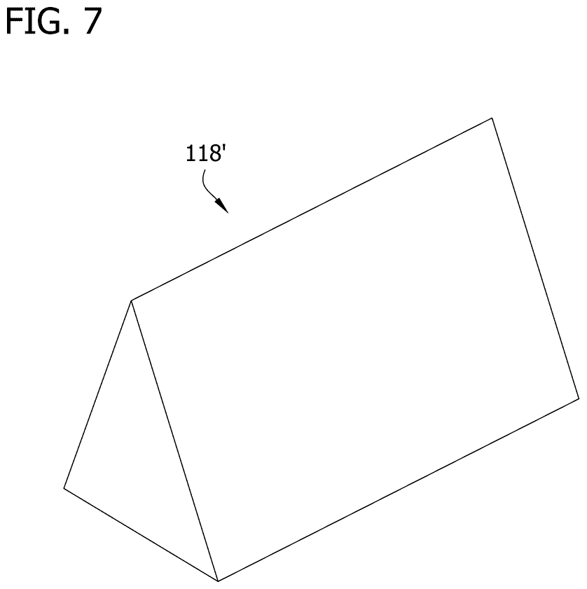

Referring to FIG. 6, the insertion stops 116 of the illustrated embodiment are configured to have point contact PC with the fluid port 10, specifically the distal end surface 14. In the illustrated embodiment, each insertion stop 116 comprises a rib extending radially relative to the longitudinal axis LA. Each insertion stop 116 includes a curved surface. The curved surface tapers (e.g., the insertion stop narrows) as the insertion stop 116 extends radially outward from the longitudinal axis LA. More specifically, the generatrix line G of the curved surface at the vertex of the curved surface is disposed at an angle α to a lateral or radial axis RA (which is perpendicular to the longitudinal axis LA). In other words, the distal end surface 14 is orientated perpendicular to the longitudinal axis LA (when the port connector 100 is connected to the fluid port 10) and the generatrix line G of the curved surface at the vertex of the curved surface is disposed at the angle α to the distal end surface. This results in the curved surface of the insertion stop forming a point contact PC with the inner edge of the distal end surface 14 that defines the fluid port outlet 16. In one embodiment, the angle α is within an inclusive range of 3 degrees to 15 degrees. In the illustrated embodiment, the angle α is about 5 degrees. Other configurations of the insertion stops 116 may be used without departing from the scope of the present disclosure. For example, in one embodiment, the curved surface of the insertion stop may not taper, and thus the curved surface will form a line contact with the fluid port 10. In other embodiments, instead of a curved surface, the insertion stop may include a beveled profile (e.g., single or double beveled edge). An example of such an insertion stop 118′ is shown in FIG. 7. The beveled profile can be angled or parallel to the distal end surface 14 (as described above with respect to the curved surface) to form a point contact or a line contact, respectively. In other embodiments, the insertion stop 116 may comprise a cone, pyramid, or any other shape having a point to form a point contact with the fluid port 10. The insertion stops 116 may have other shapes/configurations for forming point or line contacts without departing from the scope of the present disclosure, some of which are described herein.

The connector body 102 may also include one or more (e.g., a plurality of) alignment projections 118. In the illustrated embodiment, the connector body 102 includes four alignment projections 118 equally spaced part circumferentially around the longitudinal axis, although more or fewer alignment projections are within the scope of the present disclosure. Each alignment projection 118 is arranged to be disposed in the lumen 12 when the port connector 100 is connected to the fluid port 10. In the illustrated embodiment, the alignment projections 118 are mounted on the insertion portion 104. Each alignment projection 118 comprises a rib. The ribs extends radially outward from an exterior surface (e.g., exterior cylindrical surface) of the insertion portion 104. Each rib extends along the insertion portion 104, generally parallel to the longitudinal axis LA. In the illustrated embodiment, each alignment projection 118 extends proximally from one of the insertion stops 116, although in other embodiments the alignment projections and the insertion stops may be offset from one another. The alignment projections 118 help center the insertion portion 104 in the lumen 12 of the fluid port 10. Further, the alignment projections 118 are also arranged to engage an interior of the fluid port 10 to resist withdrawal of the insertion portion 104 from the lumen 12. When the connection portion 106 is connected to a hose or conduit of the sterilization apparatus, the hose may impart a lateral force to the port connector 100 causing the port connector to twist within the fluid port 10, potentially pulling the port connector out of the fluid port. The alignment projections 118 are arranged about the insertion portion 104 so that any lateral force applied by the hose will cause at least one alignment projection 118 to engage the interior of the fluid port 10, and thereby resist the applied lateral force to inhibit the port connector 100 from inadvertently withdrawing or disconnecting from the fluid port. In the illustrated embodiment, the interior of the fluid port 10 is defined by one or more cylindrical inner surfaces bounding the lumen 12. Because each alignment projection 118 may engage the fluid port 10, the alignment projections are each shaped to have only line contact or point contact with the fluid port 10, for the reasons described herein. The alignment projections 118 are not shaped to have face-to-face contact with the fluid port 10. Each alignment projection 118 can be shaped as described herein in order to form a line or point contact. For example, the alignment projections 118 can includes a curved surface (as described above) or a beveled profile (as described above) to have only line contact with the fluid port. In the illustrated embodiment, each alignment projection 118 includes a curved surface. In this embodiment, the generatrix line G of the curved surface at the vertex of the curved surface is parallel to the longitudinal axis LA. As a result, the generatrix line G of the curved surface at the vertex of the curved surface will be generally parallel to the interior surface(s) of the fluid port 10 when the insertion portion 104 is disposed in the lumen 12. The alignment projections 118 may have other shapes/configurations for forming point or line contacts without departing from the scope of the present disclosure, some of which are described below.

The connector body 102 is designed so that only parts of the connector body that engage or can engage the fluid port 10 are the insertion stops 116 and the alignment projections 118. Accordingly, the connector body 102 is configured to only have line contact and/or point contact with the fluid port 10. In other words, the connector body 102 is configured to not have any face-to-face contact with the fluid port 10. As a result, even though the port connector 100 is connected to the fluid port 10 and the connector body 102 engages the fluid port, the fluid port is still able to be completely sterilized.

In one embodiment, the connector body 102 is an integrally formed, one-piece component. For example, the connector body 102 may be made by plastic molding. Accordingly, the connector body 102 is solid, is impervious to fluid, and is free of fluid passageways except for the fluid passageway 112. In other embodiments, the connector body 102 may be made from two or more pieces joined together.

The port connector 100 may include a porous member 120 (broadly, a fluid port interface member). The porous member 120 is supported by the insertion portion 104 of the connector body 102. Specifically, the porous member 120 is mounted on the insertion portion 104. As shown in FIG. 6, the porous member 120 is arranged to engage the fluid port 10 (e.g., an interior of the fluid port) when the port connector 100 is connected to the fluid port. Specifically, the porous member 120 is arranged to engage one or more inner surfaces of the fluid port 10 that bound the lumen 12. The porous member 120 is arranged to be inserted into the lumen 12 via the fluid port outlet 16 with insertion portion 104. The porous member 120 has a porous structure defining a plurality of randomly arranged interconnected interstitial spaces that form a plurality of minute passageways 122 (FIG. 6) through and/or within the porous member. The porous member 120 engages the inner surface(s) of the fluid port 10 when the port connector 100 is attached to the fluid port. The porous member 120 is arranged relative to the connector body 102 such that at least a portion of the minute passageways 122 fluidly couple the exterior environment of the port connector 100 to the inlet 108 of the connector body, via the fluid port outlet 16, when the port connector is connected to the fluid port 10. Accordingly, when the port connector 100 is attached to the fluid port 10, porous member 120 does not form an absolute fluid tight seal with the fluid port. Instead, fluid is able to move through the porous member 120 via the minute passageways 122. However, the port connector 120 may still be considered to form a fluid tight seal with the fluid port 10. The term “fluid tight seal” as used herein refers to a seal that creates a sufficient impediment to the flow of fluid such that fluid flows from other areas (e.g., into the end of interior lumen(s) of the article opposite the fluid port) as a result of a pressure differential and does not require an absolute fluid tight seal such that no fluid can pass. For instance, the porous member 120 sufficiently impedes the flow of fluid therethrough such that fluid flows from other areas (e.g., into the end of the interior lumen(s) of the article opposite the fluid port) as a result of a pressure differential, as explained in more detail below.

The porous member 120 is preferably made of biocompatible, hydrophobic, and/or non-flammable material. In one embodiment, the porous member 120 is made of expanded polytetrafluoroethylene (ePTFE), such as FluroFlex® ePTFE, although other suitable materials may be used without departing from the scope of the present disclosure. The porous member 120 is resiliently deformable. In one embodiment, the porous member 120 may have a density within the inclusive range of about 0.3-0.6 g/cm³, or more preferably, within the inclusive range of about 0.4-0.5 g/cm³. In one embodiment, as illustrated in FIG. 6, the minute passageways 122 of the porous member 120 are generally randomly disposed throughout the entirety of the porous member.

As mentioned above, the porous member 120 engages the interior of the fluid port 10 once the insertion portion 104 is inserted into the lumen 12 of the fluid port. To ensure the porous member 120 engages the interior of the fluid port 10, the connector body 102 includes a porous member brace or wedge 124 (broadly, projection). The porous member brace 124 is supported by (e.g., mounted on) the insertion portion 104 of the connector body 102. In the illustrated embodiment, the porous member brace 124 comprises a ring extending circumferentially around the exterior of the insertion portion 104. The ring (broadly, the porous member brace 124) extends radially outward from the exterior surface of the insertion portion 104. The porous member 120 is biased outward by the porous member brace 124 so that the porous member engages the interior of the fluid port 10. In particular, the porous member brace 124 is arranged to bias only a portion of the porous member 120 outward so that said portion of the porous member engages the interior of the fluid port 10. The porous member brace 124 forms a bulge 126 (e.g., circumferentially bulge) in the porous member 120. At least a portion of the bulge 126 (broadly, at least a portion of the porous member 120) is disposed radially outward of the alignment projections 118. In the illustrated embodiment, the porous member brace 124 engages the porous member 120. The porous member brace 124 engages an interior surface of the porous member 120 to push a portion of the porous member radially outward to form the bulge 126. In the illustrated embodiment, the porous member 120 comprises a porous sleeve that encircles the insertion portion 104 (e.g., a portion thereof). The porous sleeve encircles the ring. The porous sleeve is resiliently deformable, thereby allowing the porous sleeve (broadly, the porous member 120) to be deformed (e.g., be squeezed radially inward) by the fluid port 10 when the porous member is inserted into the lumen 12 of the fluid port. Moreover, as a result of the porous member 120 being resiliently deformable, the exterior of the porous member generally contours or conforms to the exterior shape of the insertion portion 104 and porous member brace 124. This is how the porous member brace 124 forms the bulge 126 in the porous member 120. Further, the insertion portion 104 includes a tapered portion (generally at the proximal end thereof). The porous member 120 overlies the tapered portion of the insertion portion 104, thereby conforming to the shape of the tapered portion to have a tapered portion itself. The tapered portion of the porous member 120 is arranged to facilitate insertion of the porous member into the fluid port outlet 16.

The porous member 120 is configured to form an interference or friction fit with the interior of the fluid port 10. The bulge 126 forms the radially outer most extent of the porous member, with the diameter of the porous member at the bulge being greater than the diameter (e.g., inner diameter) of the lumen 12. This ensures the porous member 120 (e.g., the bulge 126) will engage and be deformed (e.g., resiliently deformed) by the fluid port 10, with the resilient deformation applying a force against the fluid port to hold the port connector 100 to the fluid port.

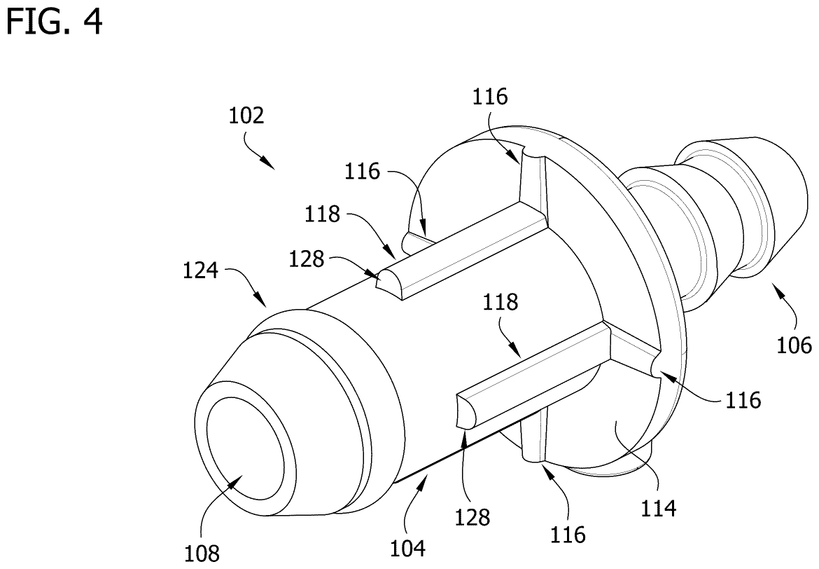

The connector body 102 may include one or more porous member stops 128 (FIG. 4). The porous member stops 128 are arranged to inhibit the porous member 120 from moving relative to the insertion portion 104. In the illustrated embodiment, the porous member stops 128 to inhibit the porous member 120 from moving relative to the insertion portion 104 in a direction (generally parallel to the longitudinal axis LA) generally toward the outlet 110 of the connector body 102. In the illustrated embodiment, the porous member stops 128 are formed by the alignment projections 118, specifically, the proximal end surfaces or faces of the alignment projections. The porous member stops 128 inhibit the porous member 120 from sliding along the insertion portion 104 as the insertion portion is inserted into the fluid port 10.

The porous member 120 permits the sterilization fluid in the environment surrounding the fluid port 10 of the article to come into contact with and sterilize the interior surfaces of the fluid port. When the negative pressure differential is applied via the negative pressure source to the port connector 100, the sterilization fluid moves (e.g., is drawn) from the exterior environment, through the fluid port outlet 16, into the lumen 12, and through the porous member 120 (specifically, through at least some of the minute passageways 122). Some of these minute passageways 122 lead to and/or extend along the interior of the fluid port 10 the porous member 120 is engaged with. As a result, as the sterilization fluid moves through the porous member 120, the sterilization fluid comes into contact with the fluid port 10, thereby sterilizing the portion of the fluid port engaged by the porous member. When the port connector 100 is inserted into the lumen 12 of the fluid port 10, the insertion stops 116 and alignment projections 118 form gaps between the fluid port and the connector body 102. The sterilization fluid flows through these gaps as the sterilization fluid flows from the exterior environment, through the fluid port outlet 16 and into the porous member 120.

In operation, to sterilize an article having the fluid port 10, the port connector 100 is connected to the fluid port by inserting the insertion portion 104 into the lumen 12. As mentioned above, the porous member 120 engages the interior of the fluid port 10. The negative pressure source of the sterilization apparatus is connected to the port connector 100 by attaching a hose to the connection portion 106. The article with the fluid port 10 is placed in a chamber (e.g., cleaning chamber). A fluid (e.g., sterilization fluid) is supplied or introduced into the chamber. The fluid may remain in the chamber for a period of time, such as 5-10 minutes, before applying the negative pressure. During this time, the fluid may naturally move or be forcefully moved around the chamber and come into contact with and sterilize surfaces of the article and fluid port, such as exposed surfaces. The fluid may also move into and through the porous member 120. Then, the operator applies the pressure differential (e.g., the negative pressure differential) via the negative pressure source. As a result, the negative pressure differential moves (e.g., draws) the sterilization fluid through the porous member, from the chamber, into the lumen 12 of the fluid port 10. As the sterilization fluid moves through the minute passageways 122 of the porous member 120, the sterilization fluid comes into contact with the surfaces engaged by the porous member, thereby sterilizing these surfaces of the fluid port 10. In addition, the negative pressure differential moves (e.g., draws) the sterilization fluid into and through the interior lumen(s) of the article and through the fluid port 10 and the port connector 100, thereby sterilizing the interior of the article. The movement of the sterilization fluid through the article and through the porous member 120 occur generally simultaneously. The sterilization fluid drawn through the article and the porous member 120 is then drawn through the connector body 102 and moves toward the negative pressure source. Thus, even though the port connector 100 is attached to the fluid port 10 during the sterilization process, the entire fluid port is exposed to the sterilization fluid and is sterilized.

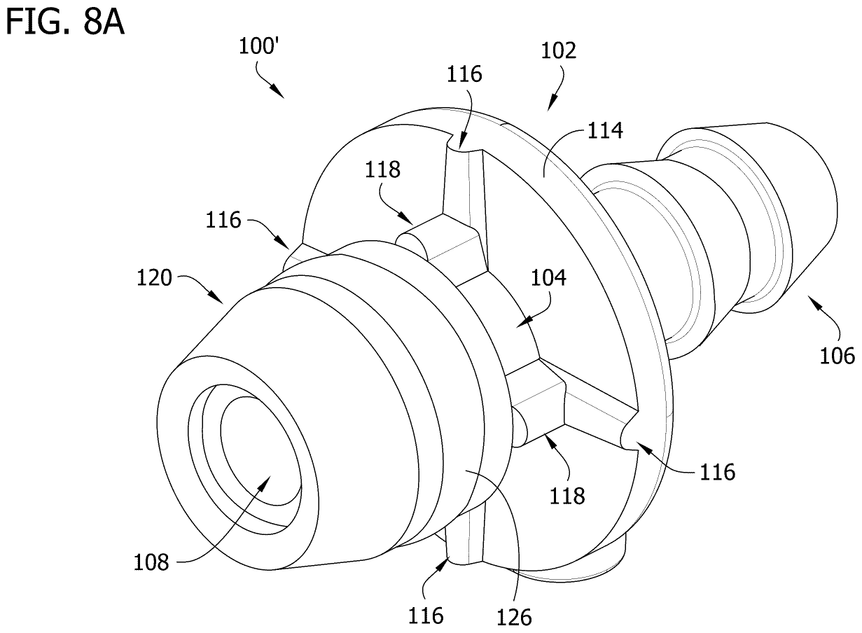

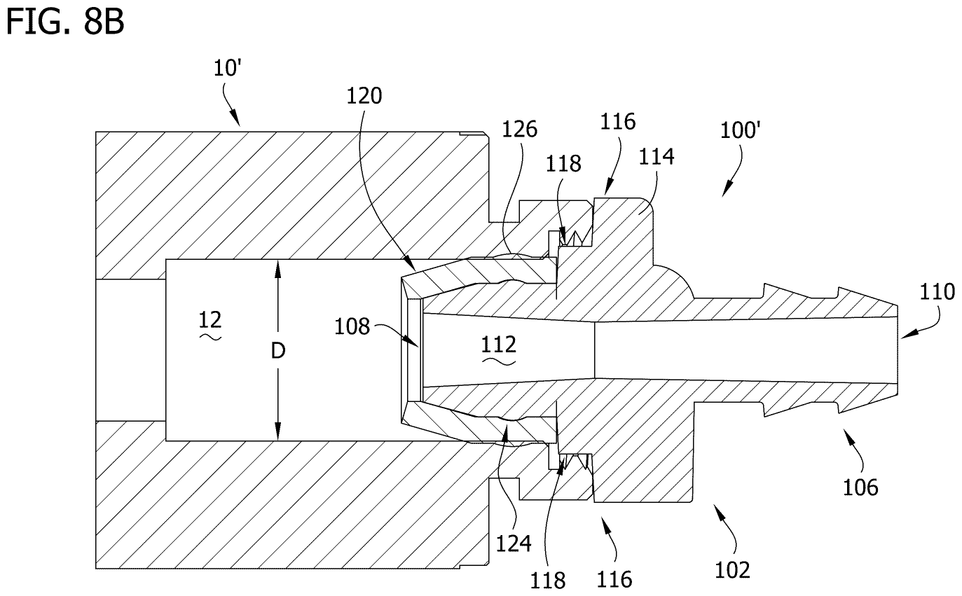

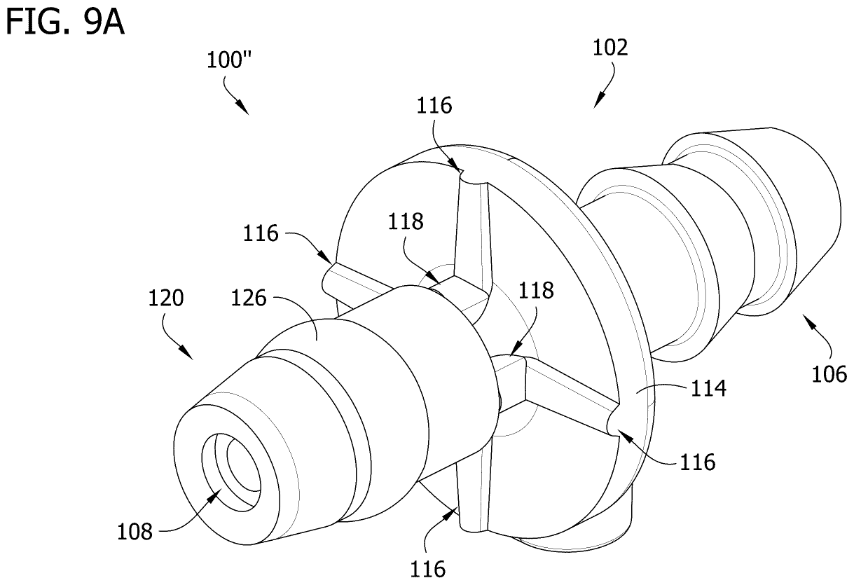

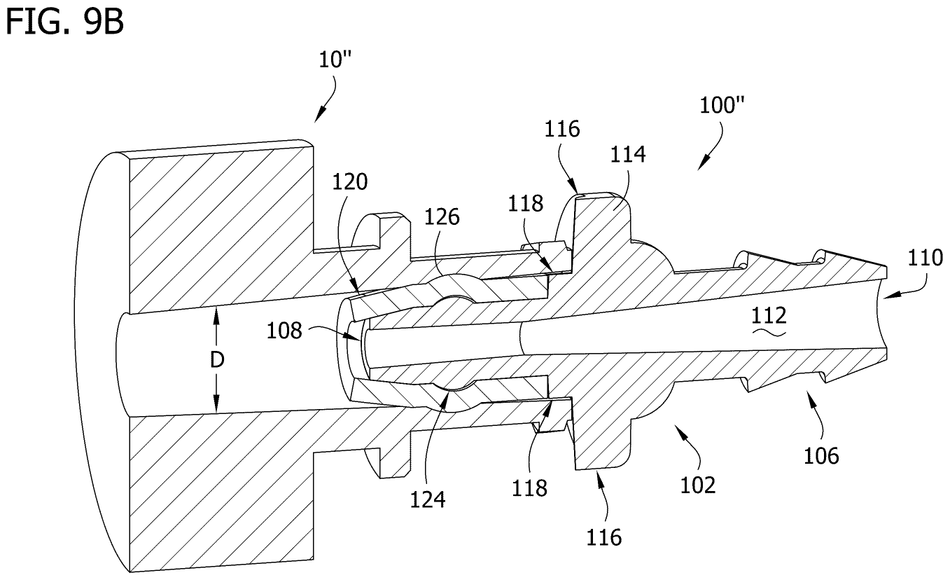

The proportions of the port connector 100 and elements thereof can be adjusted as needed to conform the port connector to different sizes of fluid ports 10. For example, the port connector 100 illustrated in FIGS. 1 and 3-6 is suitable for use with fluid ports 10 having an inner diameter D (FIG. 2) of about 7.3 mm to about 7.5 mm. FIGS. 8A-B illustrate a port connector 100′ suitable for use with fluid ports 10′ having an inner diameter D of about 5.69 mm to about 6.59 mm. FIGS. 9A-B illustrate a port connector 100″ suitable for use with fluid ports 10″ having an inner diameter D of about 4.28 mm. It is understood the port connector may be proportioned to fit fluid ports having other inner diameters without departing from the scope of the present disclosure. In one embodiment, the port connectors 100, 100′, 100″ are color coded to visually indicate the different size of fluid port 10, 10′, 10″ they are designed to fit. For example, the connector bodies of each port connector 100, 100′, 100″ can be a different color.

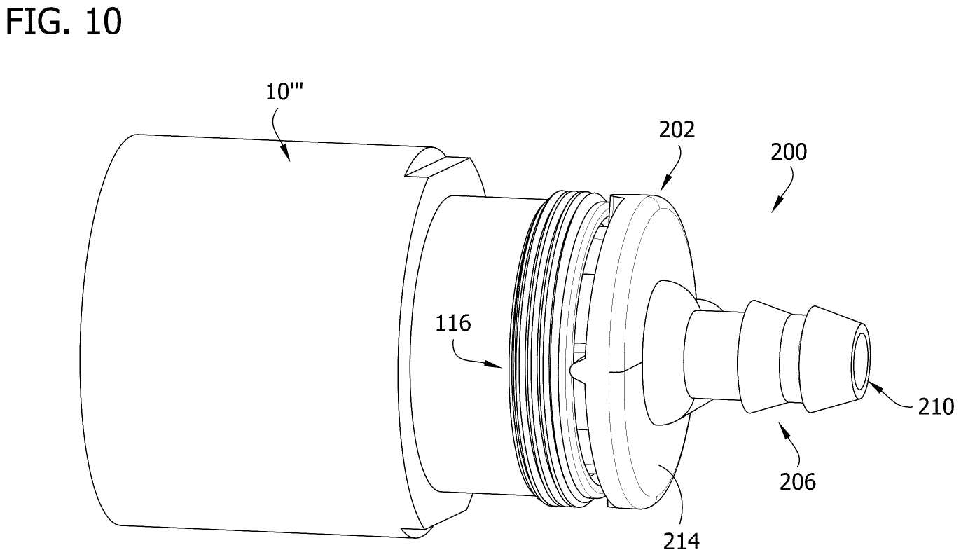

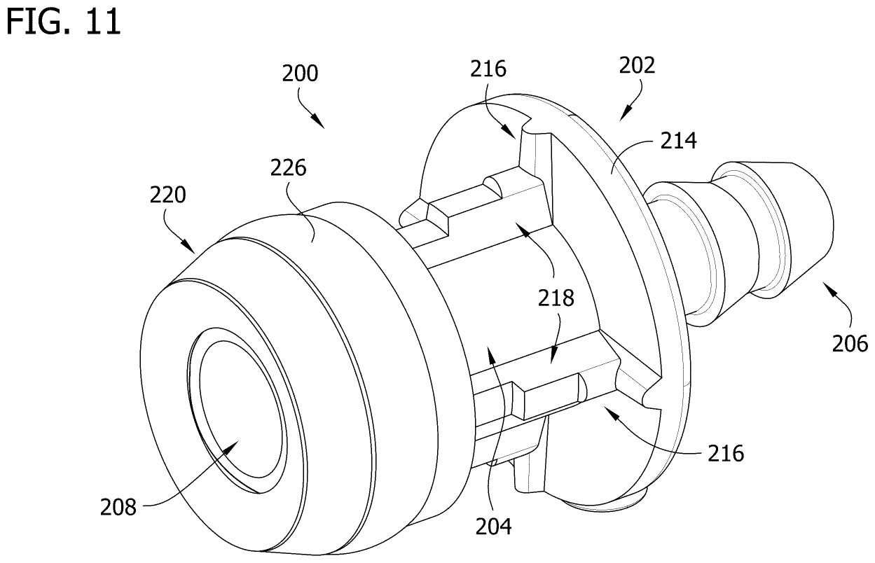

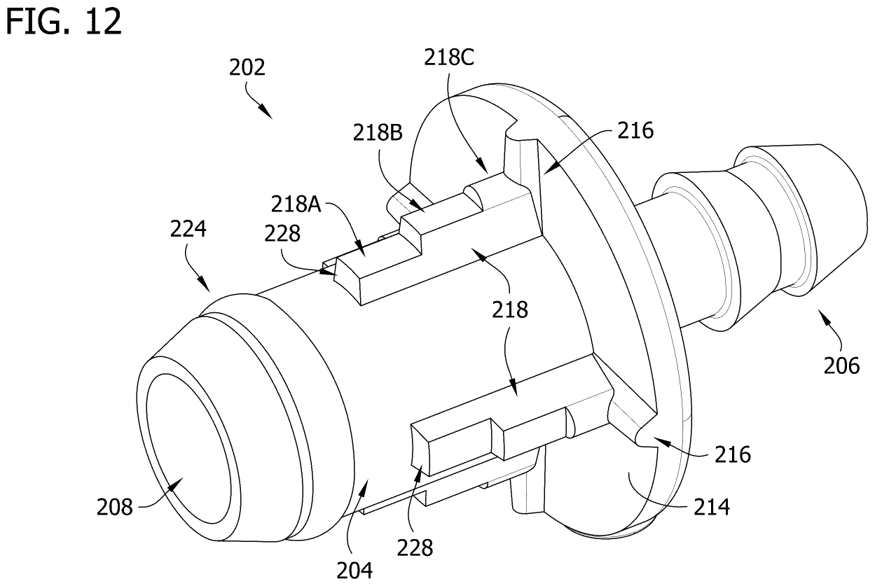

Referring to FIGS. 10-15, another embodiment of a port connector according to the present disclosure is generally indicate by reference numeral 200. The port connector 200 of FIGS. 10-15 is generally analogous to the port connector 100 of FIGS. 1-6 and thus, for case of comprehension, where similar, analogous or identical parts are used, reference numerals “100” units higher are employed. Accordingly, unless clearly stated or indicated otherwise, the above descriptions regarding the port connector 100 of FIGS. 1-6 also apply to the port connector 200 of FIGS. 10-15.

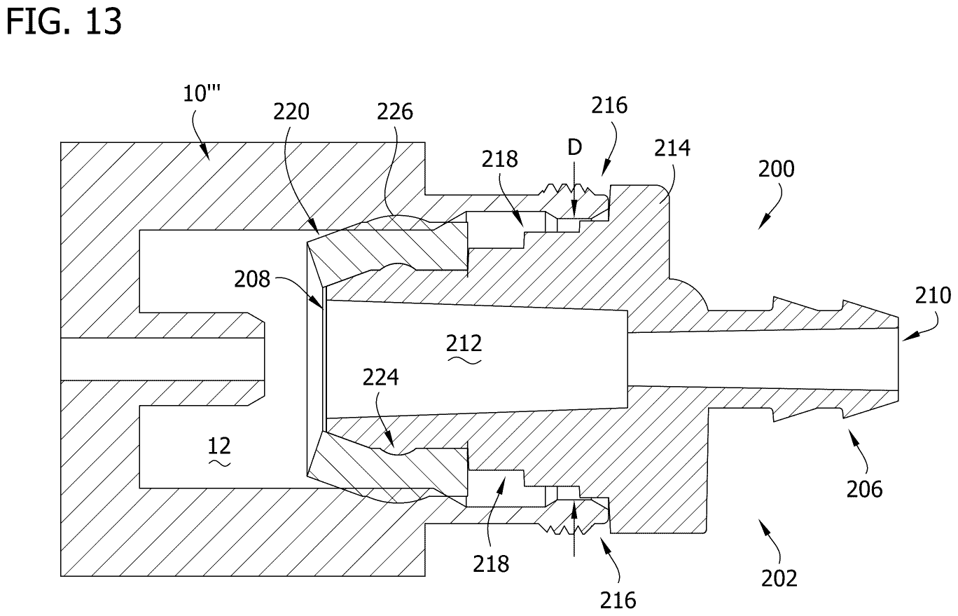

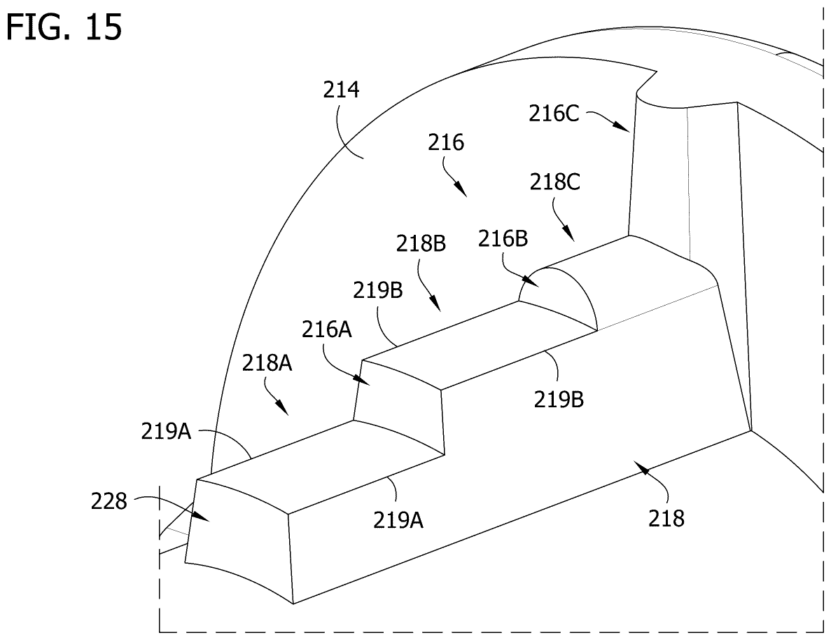

In this embodiment, the port connector 200 is configured to fit a variety of different fluid port sizes. For example, in one embodiment, the port connector 200 is suitable for use with fluid ports having an inner diameter D of about 8.3 mm to about 10.5 mm. The illustrated fluid port 10′″, that the port connector 200 is shown connected to, has an inner diameter D (FIG. 13) toward the upper end of that range (e.g., about 10.5 mm). To accommodate the different sizes of fluid ports, the alignment projections 218 (e.g., ribs) of the connector body 202 comprise a plurality of steps (e.g., alignment portions). The plurality of steps is configured to engage fluid ports of different internal diameters D. In the illustrated embodiment, each alignment projection 218 includes three steps 218A-C, although the alignment projections may include more or fewer steps without departing from the scope of the present disclosure. In one embodiment, the first step 218A is configured for use with fluid ports having an inner diameter D (e.g., a first or small inner diameter) of about 8.3 mm to less than 9.4 mm, the second step 218B is configured for use with fluid ports having an inner diameter (e.g., a second or intermediate inner diameter) of 9.4 mm to about 10 mm, and the third step 218 is configured for use with fluid ports having an inner diameter (e.g., a third or larger inner diameter) of 10 mm to about 10.5 mm. Each step 218A-C is shaped to have only line contact or point contact with the fluid port 10. For example, each step 218A-C may have a curved surface as described above with respect to alignment projection 118. In the illustrated embodiment, the third step 218C includes a curved surface as described above with respect to alignment projection 118. Instead of the curved surface, the first and second steps 218A-B include a planar surface or an extremely shallow (e.g., 5 degrees or less) beveled (e.g., double beveled) surface (FIG. 15). Because the interior (e.g., inner surface(s)) of fluid ports is curved (e.g., cylindrical), this configuration of the first and second steps 218A-B results in the edges 219A, 219B of the respective first and second steps engaging the interior surface(s) of the fluid port 10 that bound the lumen 12, thereby forming a line contact with the fluid port. It is understood that the steps 218A-C can be shaped as described herein in order to form a line or point contact. Moreover, the steps 218A-C can be shaped in the same manner or in different manners, or a combination thereof as illustrated. The steps 218A-C may have other shapes/configurations for forming point or line contacts without departing from the scope of the present disclosure.

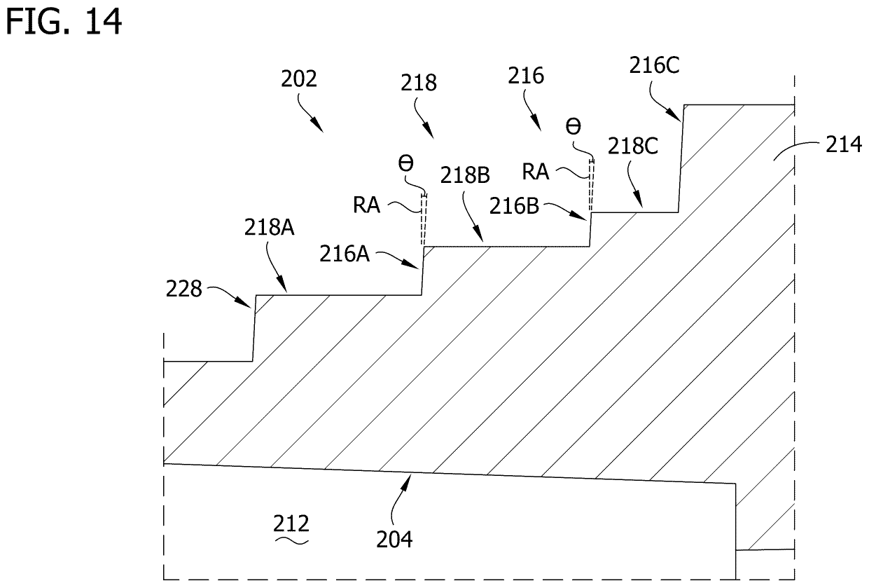

The connector body 202 also includes a series of different sets of insertion stops 216 for the different sizes of fluid ports (broadly, at least one insertion stop for each size of fluid port). In the illustrated embodiment, the connector body 202 includes three sets of insertions tops 216A-C, although the connector body may include more or fewer insertion stops without departing from the scope of the present disclosure. The third set of insertion stops 216C (e.g., third insertion stops) are generally analogous to the insertions stops 116 described above. The third insertion stops 216C are arranged to engage the distal end surface 14 of the larger or third size of fluid port 10′″. The first set of insertion stops 216A (e.g., first insertion stops) are arranged to engage the distal end surface of a smaller or first size of fluid port. The second set of insertion stops 216B (e.g., second insertion stops) are arranged to engage the distal end surface of an intermediate or second size fluid port. Although described as engaging the distal end surfaces of fluid ports, it is understood the insertion stops of the present disclosure may engage other parts of the fluid ports, such as internal lips or shoulders, to stop further insertion of the port connectors 100, 200. Like the third insertion stops 216C, the first and second insertion stops 216A, 216B are arranged to engage the fluid port to stop further insertion of the insertion portion 204 into the lumen. In the illustrated embodiment, each first insertion stop 216A is defined by one of the second steps 218B and each second insertion stop 216B is defined by one of the third steps 218C. Specifically, each first insertion stops 216A is a proximal facing end surface or face of one of the second steps 218B and each second insertion stop 216B is a proximal facing end surface or face of one of the third steps 218C. Like the third insertion stops 216C, the first and second insertion stops 216A, 216B are each shaped to have only line contact or point contact with the fluid port 10. In this embodiment, each end surface forming the first and second insertion stops 216A, 216B is planar and is disposed at an angle θ to the lateral or radial axis RA (FIG. 14). This results in the end faces of the first and second insertion stops 216A, 216B forming one or two point contacts with the circular inner edge of the distal end surface that defines the fluid port outlet of the fluid port (instead of face-to-face contact with the distal end surface). In one embodiment, the angle θ is within an inclusive range of 3 degrees to 15 degrees. In the illustrated embodiment, the angle θ is about 5 degrees. It is understood that the insertion stops 216A-C can be shaped as described herein in order to form a line or point contact. For example, the first and second insertions stops 216A, 216B can have curved surfaces. Moreover, the insertion stops 216A-C can be shaped in the same manner or in different manners, or a combination thereof as illustrated. The insertion stops 216A-C may have other shapes/configurations for forming point or line contacts without departing from the scope of the present disclosure.

It is apparent and understood that the elements, features, and/or teachings set forth in each embodiment disclosed herein are not limited to the specific embodiment(s) the elements, features, and/or teachings are described in. Accordingly, it is apparent and understood that the elements, features, and/or teachings described in one embodiment may be applied to one or more of the other embodiments disclosed herein.

When introducing elements of the present invention or the embodiment(s) thereof, the articles “a”, “an”, “the” and “said” are intended to mean that there are one or more of the elements. The terms “comprising”, “including” and “having” are intended to be inclusive and mean that there may be additional elements other than the listed elements.

Modifications and variations of the disclosed embodiments are possible without departing from the scope of the invention defined in the appended claims. For example, where specific dimensions are given, it will be understood that they are exemplary only and other dimensions are possible. As various changes could be made in the above constructions, products, and methods without departing from the scope of the invention, it is intended that all matter contained in the above description and shown in the accompanying drawings shall be interpreted as illustrative and not in a limiting sense.

What is claimed is:

1. A port connector for connecting to a fluid port of a device to be sterilized to a sterilization apparatus, the fluid port having a distal end defining a fluid port outlet, the fluid port defining a lumen extending proximally from the fluid port outlet, the port connector comprising:
a connector body having an insertion portion, a connection portion, and a porous member brace, the insertion portion being sized and shaped to be inserted into the lumen via the fluid port outlet, the insertion portion having an inlet arranged to be disposed in and in fluid communication with the lumen, the connection portion being configured to connect to the sterilization apparatus, the connection portion having an outlet in fluid communication with the inlet and configured to be fluidly coupled to the sterilization apparatus; and
a porous member supported by the insertion portion of the connector body, the porous member having a porous structure defining a plurality of minute passageways, the porous member being arranged to be inserted into the lumen via the fluid port outlet with the insertion portion, the porous member being biased outward by the porous member brace so that the porous member engages an interior of the fluid port.

2. The port connector of claim 1, wherein the porous member brace engages the porous member.

3. The port connector of claim 2, wherein the porous member brace extends outward from an exterior surface of the insertion portion.

4. The port connector of claim 3, wherein the porous member brace comprises a ring extending circumferentially around the insertion portion.

5. The port connector of claim 4, wherein the porous member comprises a porous sleeve encircling the ring.

6. The port connector of claim 1, wherein the porous member brace is arranged to bias only a portion of the porous member outward so that said portion of the porous member engages the interior of the fluid port.

7. The port connector of claim 1, wherein the porous member has a tapered portion arranged to facilitate insertion of the porous member into the fluid port outlet.

8. The port connector of claim 7, wherein the insertion portion of the connector body includes a tapered portion, the tapered portion of the porous member overlying the tapered portion of the insertion portion of the connector body.

9. The port connector of claim 1, wherein the porous member is arranged relative to the connector body such that at least a portion of the minute passageways of the porous member fluidly couple the inlet of the insertion portion of the connector body to an exterior environment of the port connector via the fluid port outlet when the port connector is connected to the fluid port.

10. The port connector of claim 1, wherein the porous member is configured to form an interference fit with the interior of the fluid port.

11. A port connector for connecting to a fluid port of a device to be sterilized to a sterilization apparatus, the fluid port having a distal end defining a fluid port outlet, the fluid port defining a lumen extending proximally from the fluid port outlet, the port connector comprising:
a connector body having an insertion portion and a connection portion, the insertion portion being sized and shaped to be inserted into the lumen via the fluid port outlet, the insertion portion having an inlet arranged to be disposed in and in fluid communication with the lumen, the connection portion being configured to connect to the sterilization apparatus, the connection portion having an outlet in fluid communication with the inlet and configured to be fluidly coupled to the sterilization apparatus, the connector body having a plurality of alignment projections arranged to be disposed in the lumen and engage an interior of the fluid port to resist withdrawal of the insertion portion from the lumen; and
a porous member supported by the insertion portion of the connector body, the porous member having a porous structure defining a plurality of minute passageways, the porous member being arranged to be inserted into the lumen via the fluid port outlet with the insertion portion.

12. The port connector of claim 11, wherein each alignment projection comprises a rib.

13. The port connector of claim 12, wherein each rib extends parallel to a longitudinal axis of the connector body, the longitudinal axis extending between the inlet and the outlet of the connector body.

14. The port connector of claim 12, wherein each rib comprises a plurality of steps configured to engage fluid ports of different internal diameters.

15. The port connector of claim 11, wherein each alignment projection is shaped to have only line contact or point contact with the fluid port.

16. The port connector of claim 11, wherein each alignment projection includes a curved surface or a beveled profile configured to have only line contact with the fluid port.

17. The port connector of claim 11, wherein the connector body includes a porous member stop arranged to inhibit the porous member from moving relative to the insertion portion of the connector body in a direction generally toward the outlet of the connector body.

18. The port connector of claim 17, wherein the porous member stop is formed by one of the plurality of alignment projections.

19. The port connector of claim 11, wherein the connector body includes one or more insertion stops arranged to engage the fluid port to stop further insertion of the insertion portion of the connector body into the lumen.

20. The port connector of claim 19, wherein the one or more insertion stops are each shape to have only line contact or point contact with the fluid port.
